(19) United States
(12) Patent Application Publication
(10) Pub. No.: US 2023/0390059 A1
(43) Pub. Date: Dec. 7, 2023

(54) INTERFACE FOR PROSTHETIC CARDIAC VALVE AND DELIVERY SYSTEMS

(71) Applicant: SHIFAMED HOLDINGS, LLC, Campbell, CA (US)

(72) Inventors: Jasper Ellington ADAMEK-BOWERS, San Francisco, CA (US); Jordan SKARO, San Jose, CA (US); Jonathan OAKDEN, San Jose, CA (US); Andrew BACKUS, Santa Cruz, CA (US); Uyenchi HA, Campbell, CA (US)

(21) Appl. No.: 18/043,499

(22) PCT Filed: Aug. 24, 2021
(86) PCT No.: PCT/US2021/047224

§ 371 Date: Feb. 28, 2023

Related U.S. Application Data
(60) Provisional application No. 63/072,802, filed on Aug. 31, 2020.

(51) Int. Cl. A61F 2/24

(52) U.S. Cl. CPC A61F 2/243

(57) ABSTRACT

Interfaces for delivery systems for delivering and deploying prosthetic heart valves. A prosthetic valve may be secured to a patient's native diseased valve by a spiral-shaped anchor that encircles an outer periphery of the prosthetic valve. In some examples, the interface provides a releasable connection between the anchor and a tether that guides placement of the anchor with respect to the patients native valve. Once the anchor is properly positioned, the prosthetic valve may be positioned and expanded within the patient's native valve and the anchor. The tether-anchor interface can release the anchor from the tether, leaving the anchor and valve prosthesis secured in place within the heart.

### CROSS REFERENCE TO RELATED APPLICATIONS

This application claim priority to U.S. Provisional Patent Application No. 63/072,802, filed on Aug. 31, 2020, and entitled “INTERFACE FOR PROSTHETIC CARDIAC VALVE AND DELIVERY SYSTEMS,” which is incorporated herein by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transseptally, requiring transapical access instead.

Thus, a new valve delivery system or method that overcomes some or all of these deficiencies is desired.

### SUMMARY

Described herein are devices, systems and methods related to delivering a prosthetic heart valve into a patient's heart to replace a diseased native valve. The prosthetic valve may be secured to the patient's native valve by a spiral-shaped anchor that encircles an outer periphery of the prosthetic valve. In some cases, the anchor may be deployed within the heart separately from the prosthetic valve using an anchor delivery system. The anchor may be manipulated within the heart using one or more components of the anchor delivery system to position the anchor around at least a portion of the chordae tendineae. The anchor may be connected to a tether via a connector to maintain and/or adjust a position of the anchor. Once the anchor is properly positioned, the prosthetic valve may be positioned and expanded within the patient's native valve and the anchor. The connector can then release the anchor from the tether, leaving the anchor and valve prosthesis secured in place within the heart.

According to some aspects, a system for anchoring a valve prosthesis comprises: an anchor comprising a wire having a shape and size to engage with chordae tendineae and/or leaflets of a native valve; and a tether releasably coupled to a proximal end of the anchor, the tether configured to maintain or adjust a position of the anchor, the tether comprising a cable housed within a tubular housing, a distal end of the tubular housing having a connector with an opening sized and shaped to accept and engage with an engagement feature at the proximal end of the anchor, the connector configured to transition between a locked state to secure the anchor with the tether, and an unlocked state to decouple the anchor and the tether.

In this aspect, the wire may have a spiral shape configured to extend around a least a portion of the chordae tendineae. In this aspect, the wire may be configured to extend around the valve prosthesis. In this aspect, a distal end of the cable may comprise a corresponding engagement feature configured to engage with the engagement feature of the anchor. In this aspect, the connector may be configured to radially surround the engagement feature of the anchor and the corresponding engagement feature of the cable when the connector is in the locked state. In this aspect, the system may further comprise an actuator at a proximal end of the tether, the actuator configured to transition the connector between the locked and unlocked states. In this aspect, the anchor may be rotationally locked with respect to the tether when the connector is in the locked state. In this aspect, the anchor and the tether may be rotationally locked with respect to the tubular housing when the connector is in the locked state. In this aspect, the anchor and the tether may be rotationally locked with respect to the tubular housing when the connector is in a locked state. In this aspect, a diameter of the tether may range between 0.03 inches and 0.05 inches. In this aspect, the connector may comprise a retention wire (e.g., FIGS. 4A-4E) that runs through the tubular housing and is threaded through an opening (e.g., hole) of the anchor to engage the tether with the anchor. In this aspect, the retention wire (e.g., FIGS. 4A-4E) may be configured to disengage from the anchor when pulled proximally. In this aspect, the tubular housing may include a release latch (e.g., FIGS. 5A-5I) configured to frictionally engage with a retention wire, wherein the release latch is configured to deflect upon retraction of the retention wire to disengage the retention feature and release the anchor from the tether. In this aspect, the retention feature may include a recess (e.g., FIGS. 5A-5I) configured to entrap a cross-pin running through the tubular housing. In this aspect, the cross-pin (e.g., FIGS. may be configured to engage with a ramp defined by the recess. In this aspect, the tubular housing radially may surround a distal end of the cable and the proximal end of the anchor when the connector is in the locked state. In this aspect, a retention feature of the anchor may be configured to interlock with a corresponding retention feature of the cable within the tubular housing (e.g., FIGS. 6A-6N). In this aspect, the tubular housing may be configured to radially surround the retention features of the anchor and the cable to maintain the anchor secured to the cable in the locked state (e.g., FIGS. 6A-6N). In this aspect, a proximal portion of the tether may comprise a tension nut and a spacer, the spacer sized and shaped to fit between the tubular housing and the tension nut to form a gap therebetween and to apply a proximal tension on the cable, to retain the cable proximal to the connector at the distal end of the tubular housing when in the locked state (e.g., FIGS. 6A-6N). In this aspect, the spacer may be configured to be removed from the gap to release the proximal tension such that the cable may be advanced distally past the connector and decouple the cable from the anchor (e.g., FIGS. 6A-6N). In this aspect, the retention features of the cable and the anchor may have interface surfaces that are non-orthogonal with respect to a central axis of the cable and the anchor (e.g., FIGS. 7A-7C). In this aspect, the tubular housing may include a tab that is configured to engage with a retention feature of the cable to deflect the tab when the tubular housing is retracted proximally (e.g., FIGS. 7A-7C). In this aspect, the tubular housing may include a threaded interior surface that is configured to engage with a threaded exterior surface of the cable, wherein rotation of the cable relative to the housing causes the connector to transition between the locked and unlocked states (e.g., FIGS. 8A-8B). In this aspect, the tubular housing may include flat internal surfaces that engage with corresponding flat external surfaces of the anchor and the cable when the connector is in a locked state to prevent rotation of the anchor and the cable with respect to the tubular housing (e.g., FIGS. 7A-7C). In this aspect, the actuator may comprise a spacer configured to apply a proximal tension on an internal cable of the tether to prevent the cable from advancing distally past the connector at a distal end of the tether, thereby placing the connector in the locked state (e.g., FIGS. 6A-6N). In this aspect, the actuator may include a screw mechanism configured to transition the connector between the locked and unlocked states, the screw mechanism comprising a threaded proximal nut configured to engage with a threaded outer surface of an exterior surface of the tether (e.g., FIGS. 9A-9B and 10A-10B). In this aspect, the threaded proximal nut may comprise a coil within the proximal nut (e.g., FIGS. 10A-10B). In this aspect, the proximal end of the anchor may be configured to cooperate with a retractable wire to frictionally engage within the inner walls defining the opening of the connector, wherein proximal retraction of the retractable wire causes the retractable wire to exit the opening of the connector to disengage and release the anchor from the connector (e.g., FIGS. 11A-11C). In this aspect, the tether may be shaped and sized to guide a valve delivery catheter for placement of the valve prosthesis within the native heart.

According to some aspects, a method of delivering a valve prosthesis in a subject's heart comprises: deploying an anchor near a diseased native valve such that the anchor is engaged with chordae tendineae of the diseased native valve, wherein the anchor is secured to a tether via a connector in a locked state; delivering a frame structure of the valve prosthesis to the diseased native valve; deploying the valve prosthesis within the diseased native valve such that the anchor radially surrounds the valve prosthesis; and decoupling the anchor from the tether by transitioning the connector from the locked state to an unlocked state.

In this aspect, further comprising guiding placement of the frame structure toward the diseased native valve by the tether. In this aspect, a position of the anchor may be maintained at least in part by controlling a position of the tether. In this aspect, the position of the anchor may be an axial position. In this aspect, controlling a position of the tether ma comprise tensioning the tether (e.g., proximally). The method of claim 29, wherein transitioning the connector from the locked state to an unlocked state comprises manipulating an actuator at a proximal end of the tether. In this aspect, manipulating the actuator may include removing a spacer between the tether and a proximal nut. In this aspect, the tether may comprise a cable housed within a tubular housing, wherein manipulating the actuator further includes advancing the cable distally with respect to the tubular housing. In this aspect, the tether may comprise a cable housed within a tubular housing, wherein manipulating the actuator further includes retracting the tubular housing proximally with respect to the cable. In this aspect, the anchor may be locked within a tubular housing of the tether when the connector is in the locked state. In this aspect, the method may further comprise rotationally locking the anchor and cable with respect to the tubular housing when the connector is in the locked state. In this aspect, the method may further comprise rotationally locking the anchor with respect to the tether when the connector is in the locked state.

According to some aspects, a tether configured to maintain or adjust a position of the anchor for anchoring a valve prosthesis comprises: a cable housed within a tubular housing; and a connector at a distal end of the tether, the connector having an opening sized and shaped to accept and engage with an engagement feature of the anchor, the connector configured to transition between a locked state to secure the anchor with the tether, and an unlocked state to decouple the anchor and the tether.

In this aspect, a distal end of the cable may comprise a corresponding engagement feature configured to engage with the engagement feature of the anchor. In this aspect, the tether may further comprise an actuator at a proximal end of the tether, the actuator configured to transition the connector between the locked and unlocked states.

These and other aspects are described herein.

### BRIEF DESCRIPTION OF THE DRAWINGS

Novel features described herein are set forth with particularity in the appended claims. A better understanding of the features and advantages of the embodiments may be obtained by reference to the following detailed description that sets forth illustrative embodiments and the accompanying drawings.

FIGS. 1A-1G illustrate sequential views of an exemplary method of implanting an anchor for a valve prosthesis near a native valve annulus.

FIGS. 2A-2K illustrate sequential views of an exemplary method of delivering a valve prosthesis to an anchor previously placed near a native valve annulus.

FIG. 3A illustrates a diagram of an exemplary anchor and tether system for implanting a valve prosthesis.

FIGS. 3B and 3C illustrate an exemplary anchor wrapped around a frame structure of a prosthetic valve: FIG. 3B shows the anchor unattached to a tether; and FIG. 3C shows the anchor attached to a tether via a connector.

FIGS. 4A-4E illustrate an exemplary connection mechanism for coupling an anchor to a tether: FIG. 4A shows a side view of the connection mechanism; FIG. 4B shows a side cross-sectional view of the connection mechanism; FIG. 4C shows the anchor coupled to the tether (e.g., during deployment of the anchor to the heart); FIG. 4D shows actuation of the release assembly; and FIG. 4E shows the tether retracted into a delivery device housing and de-coupled from the anchor.

FIGS. 5A-5I illustrate another exemplary connection mechanism for coupling an anchor to a tether: FIG. 5A shows a side view of the connection mechanism; FIG. 5B shows a side cross-sectional view of the connection mechanism; FIG. 5C shows a top view of the connection mechanism; FIG. 5D shows a side view of a tube configured for coupling with the tether and having a release latch; FIG. 5E shows a top view of the tube; FIG. 5F shows a side view of a proximal end of the anchor having engagement features for engaging with the tube; FIG. 5G shows a top view of the proximal end of the anchor; and FIGS. 5H and 5I show a release sequence of the anchor from the tether.

FIGS. 6A-6N illustrate another exemplary connection mechanism for coupling an anchor to a tether: FIGS. 6A-6D show a side views of the connection mechanism transitioning from a locked to an unlocked state; FIG. 6E shows a section view of the proximal portion of the connection mechanism in a locked state; FIG. 6F shows a section view of the proximal portion of the connection mechanism in an unlocked state; FIG. 6G shows a perspective view of the distal portion of the connection mechanism in a locked state; FIG. 6H shows a section view of the distal portion of the connection mechanism in an unlocked state; FIG. 6I shows the proximal portion of the connection mechanism in a locked state; FIG. 6J shows the proximal portion of the connection mechanism in an unlocked state; FIG. 6K shows the distal portion of the connection mechanism in a locked state; FIG. 6L shows the distal portion of the connection mechanism in an unlocked state; FIG. 6M shows an image of the anchor coupled to the tether inside a heart; and FIG. 6N shows an image of the anchor uncoupled from the tether inside a heart.

FIGS. 7A-7C illustrate another exemplary connection mechanism for coupling an anchor to a tether: FIG. 7A shows a section view of the connector in a locked state; FIG. 7B shows a section view of the connector in an unlocked state; and FIG. 7C shows a perspective view of the connector in a locked state.

FIGS. 8A and 8B illustrate an exemplary connection mechanism having a screw mechanism for coupling an anchor to a tether: FIG. 8A shows a section view of the connector in a locked state; FIG. 8B shows a perspective and partially transparent view of the connector in the locked state.

FIGS. 9A-9F illustrate an exemplary actuator portion having a screw mechanism for actuating a connector: FIG. 9A shows a side view of the actuator in a state that locks the connector; FIG. 9B shows a side view of the actuator in a state that unlocks the connector; FIG. 9C shows a section view of the actuator in a locked state; FIG. 9D shows a perspective, partially transparent view of the proximal end of the actuator; FIG. 9E shows a side, partially transparent view of the proximal end of the actuator; and FIG. 9F shows a perspective, partially transparent view of the proximal end of the actuator including the mating feature.

FIGS. 10A and 10B illustrate another exemplary actuator portion having a screw mechanism for actuating a connector: FIG. 10A shows a section view of the actuator in a state that locks the connector; and FIG. 9B shows a perspective view of the actuator in a state that locks the connector.

FIGS. 11A-11C illustrate an exemplary connection mechanism having a removable shim configuration: FIG. 11A shows a side perspective view of the connection mechanism; FIG. 11B shows a front perspective view of the connection mechanism; and FIG. 11C shows a section view of the connection mechanism.

### DETAILED DESCRIPTION

Described herein are devices and methods for use in delivering a prosthetic cardiac valve system, for example during a mitral valve replacement. The prosthetic valve system can include a prosthetic valve, which is configured to replace a diseased native valve, and an anchor, which is configured to secure the prosthetic valve in place within the diseased native valve. In some cases, the anchor and the prosthetic valve are delivered to the heart separately. In some cases, the anchor is delivered to the heart using an anchor delivery catheter system and the prosthetic valve is delivered to the heart using a valve delivery catheter system. In some cases, a tether is coupled with the anchor and assists with positioning the anchor and/or with delivering the valve delivery catheter system. In other cases, the anchor and prosthetic valve are delivered to the heart using a single delivery catheter system.

The devices and method described herein relate to interfaces between the anchor and/or prosthetic valve and the delivery system(s) for delivering and deploying the anchor and/or prosthetic valve. In some examples, the interfaces provide a releasable connection between the anchor and the tether. The tether can be configured to extend out of the heart, and in some cases, out of the patient's body. In some cases, the tether may be used to guide positioning of the anchor and/or placement of the valve prosthesis within the native valve (e.g., via the valve delivery catheter). Once the anchor and valve prosthesis are properly positioned and deployed, the tether may be disconnected from the anchor and removed, along with other parts of the delivery catheter(s), leaving the anchor and valve prosthesis secured in place within the heart.

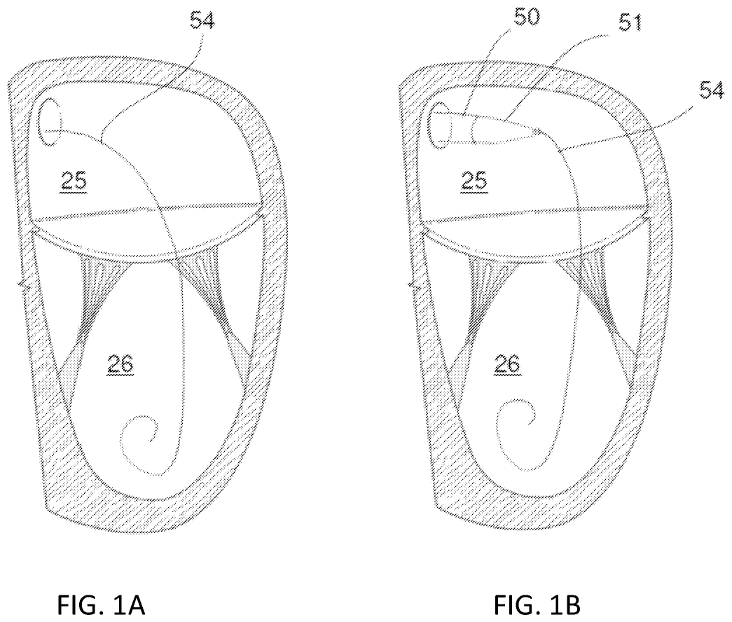

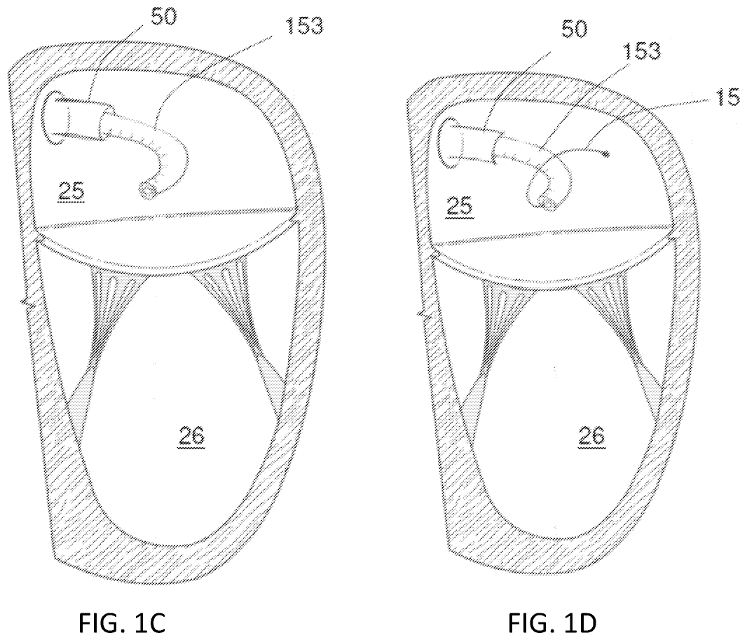

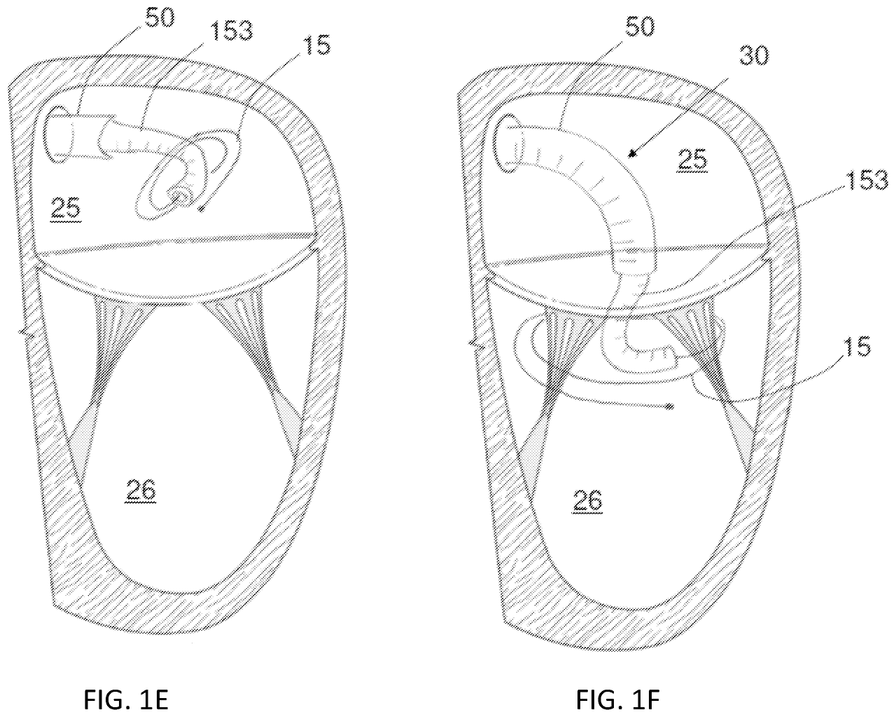

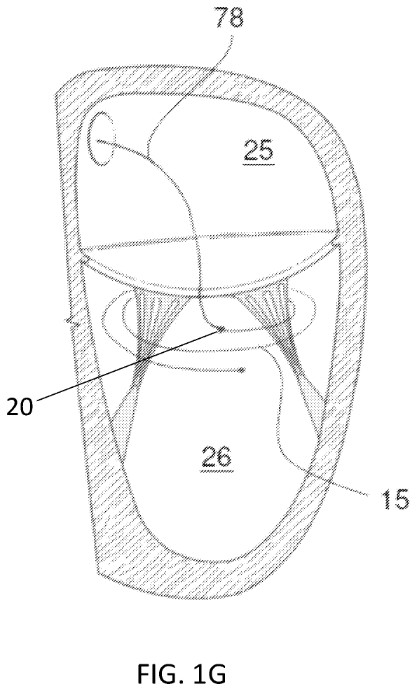

FIGS. 1A-1G show sequential views of a method of implanting an anchor 15 for a valve prosthesis using an anchor delivery system. At FIG. 1A, a transseptal puncture is made. A guidewire 54 is then routed through the puncture site and left either in the left atrium 25 or across the mitral valve into the left ventricle 26. At FIG. 1B, the outer sheath 50 (optionally with an inner dilator 51) of the anchor delivery system is tracked over the guidewire 54 until the distal end of the outer sheath 50 protrudes into the left atrium 25. The guidewire 54 and inner dilator 51 are then removed from the outer sheath 50. At FIG. 1C, the inner sheath and anchor guide 153 are inserted through the outer sheath 50 until the distal tip of the anchor guide 153 extends into the left atrium 25. The anchor guide 153 can be positioned and/or oriented as desired by steering the distal end of the sheath 50 and/or rotating the inner shaft and anchor guide 153 within the sheath 50. At FIG. 1D, once the anchor guide 153 is in the correct orientation, the anchor 15 can be pushed out through distal tip of the anchor guide 153. The anchor 15 can be, or can include, a wire that takes on a particular shape. In this example, the anchor 15 takes on a spiral shape with the wire circling around central axis and extending around the chordae tendineae. At FIG. 1E, the curvature of the anchor guide 153 can cause torsion on the anchor 15, causing the anchor 15 to deploy concentrically with the outer sheath 50 into the atrium 25. At FIG. 1F, the entire delivery system can be pushed and steered (for example, via steering mechanisms in the outer sheath 50) towards an apex of the ventricle 26, crossing through the mitral valve. In some embodiments, counter-rotation of the anchor 15 (via counter-rotation of the inner shaft and guide 153) may aid in getting the anchor across the mitral valve without tangling. Once the anchor 15 is at the correct depth within the ventricle 26, forward rotation of the anchor 15 (via forward rotation of the inner shaft and guide 153) will allow the anchor 15 to encircle the mitral leaflets and chordae. In some embodiments, the anchor 15 can be deployed towards the apex to avoid interference with mitral leaflet motion. At FIG. 1G, the outer sheath 40, inner sheath, and anchor guide 153 are removed, leaving a tether 78 in place (and still attached at connector 20 to the anchor 15). A frame structure of the valve prosthesis can then be delivered over the tether 78 and into place within the mitral valve using a valve delivery system.

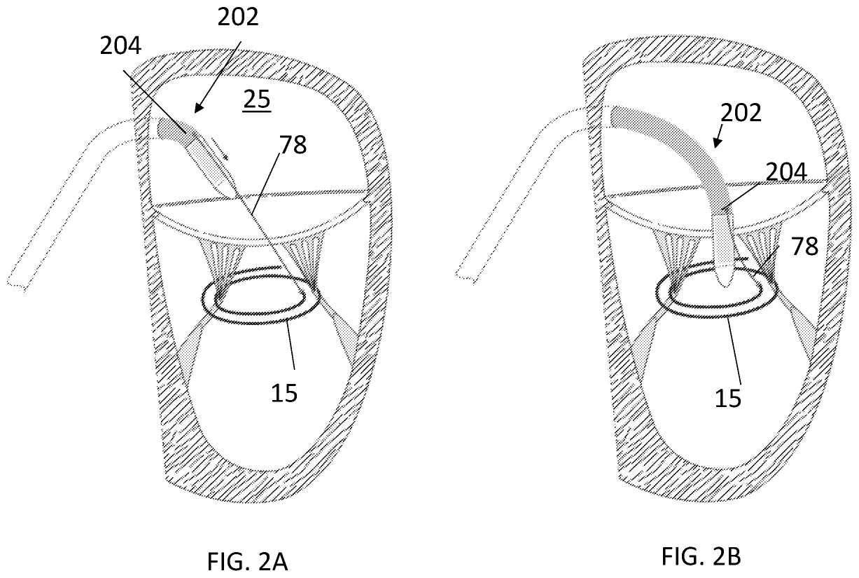

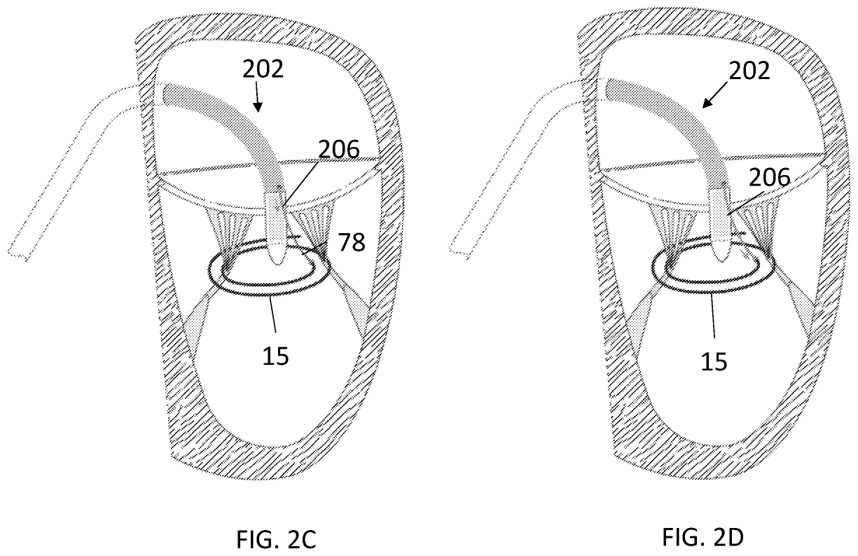

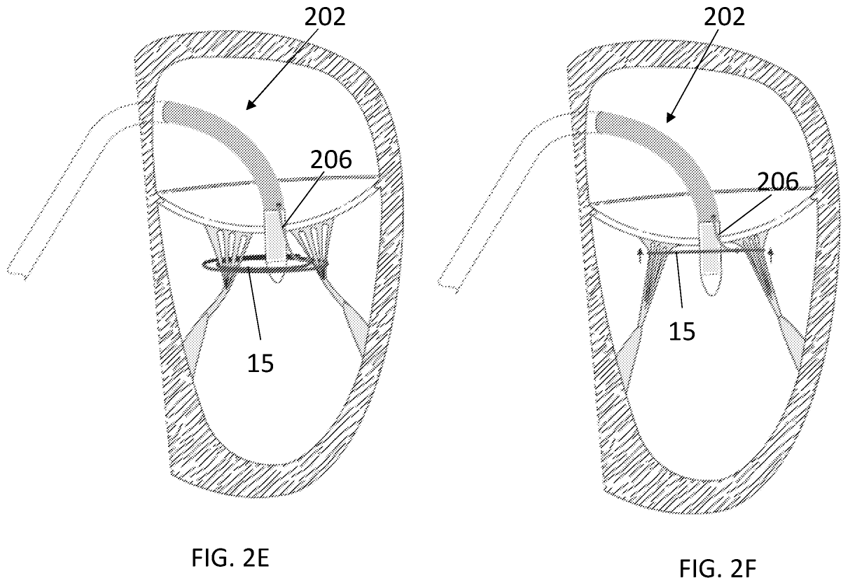

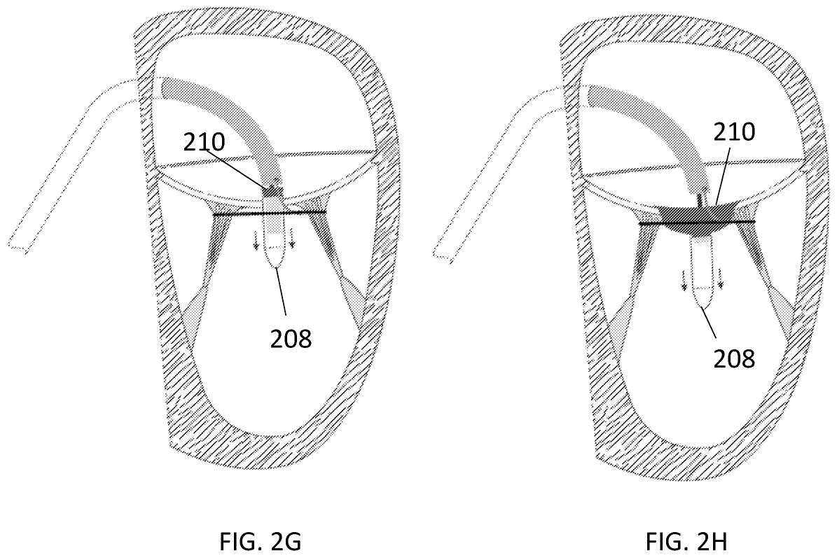

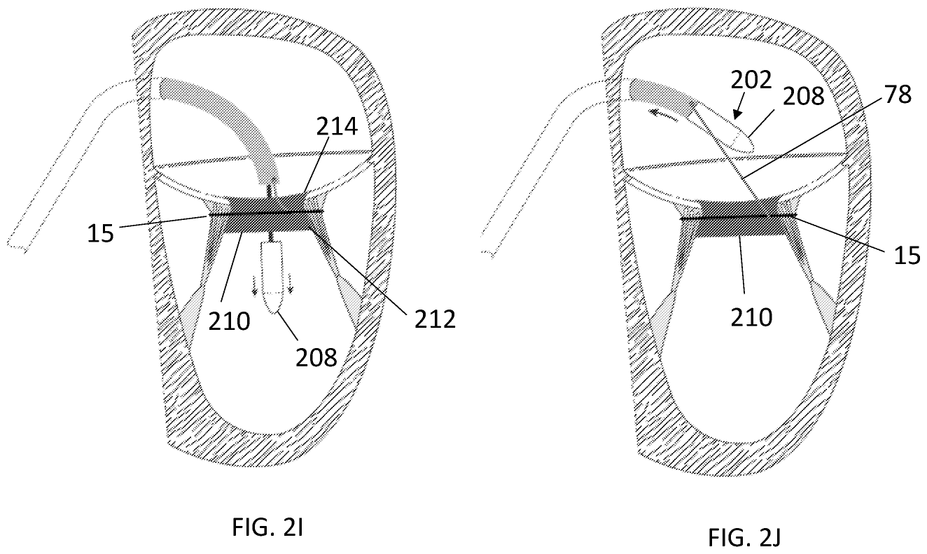

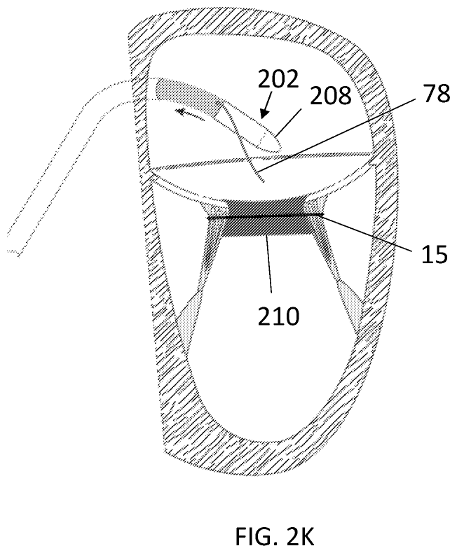

FIGS. 2A-2K show an example method of delivering a prosthetic valve using a valve delivery system to an anchor having a tether, after the anchor has already been placed (e.g., as shown in FIG. 1G). A proximal portion (not shown) of the tether 78, that is external to the patient, can be loaded into a valve delivery system 202 (e.g., via port 204) to enable the valve delivery system 202 to be tracked thereover and into the left atrium 25 (e.g., as shown in FIG. 2A). The tether 78 can guide and/or promote a positional relationship between anchor 15 and valve delivery system 202. As shown in FIG. 2B, the valve delivery system 202 is pushed across the mitral valve. In this example, the tether 78 can be positioned within a (e.g., separate) lumen within the valve delivery catheter and exit through a side port 204, as shown in FIGS. 2A and 2B. Other embodiments for carrying the tether 78 are also possible. For example, the valve delivery catheter may include a monorail for carrying the tether 78. In other cases, the tether 78 may be carried within an inner shaft of the valve delivery catheter.

FIG. 2C shows a positioning tool 206 being tracked over the tether 78. Referring to FIG. 2D, the positioning tool 206 is advanced distally over the tether 78 until a distal end of the positioning tool 206 meets a proximal end of the anchor 15. The positioning tool 206 can be positioned or steered in a way to transition the positioning tool 206 from a first (e.g., flexible) configuration to a second (e.g., stiffened) configuration. In the second configuration, the positioning tool 206 can be used to maintain or adjust a position the anchor 15. For example, the positioning tool 206 can be used to achieve axial alignment between the anchor 15 and the valve delivery system and perpendicular alignment to the native valve. The positioning tool 206 can also be used to pull the anchor up, as shown in FIGS. 2E and 2F, towards mitral annulus to better engage with mitral leaflets and reduce leakage around valve/anchor (paravalvular leakage). FIG. 2F shows the anchor in position near the mitral annulus. The positioning tool 206 can be used to position the anchor 15 in a way that achieves planarity with the mitral annulus, which can help ensure planar positioning of the prosthetic valve frame with respect to the mitral annulus. The positioning tool 206 can also ensure that the anchor 15 maintains good encircling of the native anatomy (e.g., chordae and leaflets).

Referring to FIG. 2G, once the anchor 15 is properly positioned, a valve capsule 208 can be translated distally, exposing the prosthetic valve frame 210. FIG. 2H shows the valve capsule 208 continuing to move distally, exposing more of the prosthetic valve frame 210. As more of the prosthetic valve frame 210 is exposed, the exposed portions may begin to expand, as depicted in FIG. 2H. A deployment mechanism can maintain an axial height relationship between the prosthetic valve 210, anchor 15, and the native valve anatomy. For example, a steering mechanism of the valve sheath of the valve delivery system can be used to control directional position.

Referring to FIG. 2I, the valve capsule 208 continues to translate distally, until the entire valve frame 210 is exposed. The valve frame 210 is held in place using a compression fit between the valve frame 210 and the anchor 15. As shown in FIGS. 2H and 2I, the valve frame 210 may comprise a ventricular flare 212 and an atrial flare 214 with a waist positioned around a midsection of the valve frame 210. The valve frame 210 may seat against the anchor 15 such that the anchor 15 surrounds the waist section of the valve frame 210. FIG. 2J shows the valve delivery system 202 being retracted and removed from the center of the valve frame 210. FIG. 2K shows the tether 78 detached from the anchor 15 and partially retracted into the valve delivery system 202. The valve delivery system 202 (including the valve capsule 208, positioning tool 206 and the tether 78) can then be withdrawn and removed from the body, leaving the anchor 15 and prosthetic valve 210 in place.

The anchor deployment methods shown in FIGS. 1A-1G and the valve deployment methods shown in FIGS. 2A-2K are presented only as examples. Any of a number of anchor deployment and valve deployment methods may be used in accordance with the devices and methods described herein. Example valve prostheses and related aspects are described in International Application No. PCT/US2020/027744, filed on Apr. 10, 2020, published as WO 2020/210685, and entitled “MINIMAL FRAME PROSTHETIC CARDIAC VALVE DELIVERY DEVICES, SYSTEMS, AND METHODS,” which is incorporated herein in its entirety for all purposes. Example anchors and related aspects are described in U.S. application Ser. No. 16/723,537, filed on Dec. 10, 2019, published as U.S. Patent Application publication No. US20200261220A1, and entitled “PROSTHETIC CARDIAC VALVE DEVICES, SYSTEMS, AND METHODS,” which is incorporated herein in its entirety for all purposes. When the valve prosthesis and anchor 15 are fully deployed and in the correct anatomical positions, the tether 78 can be released from the anchor 15 via a connector 20.

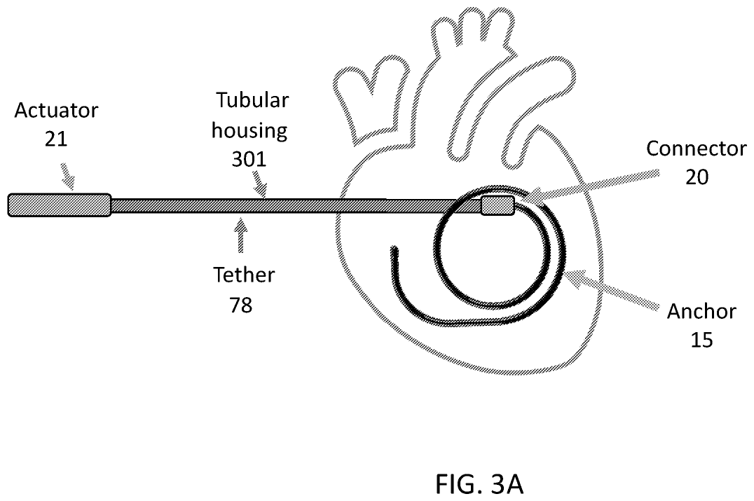

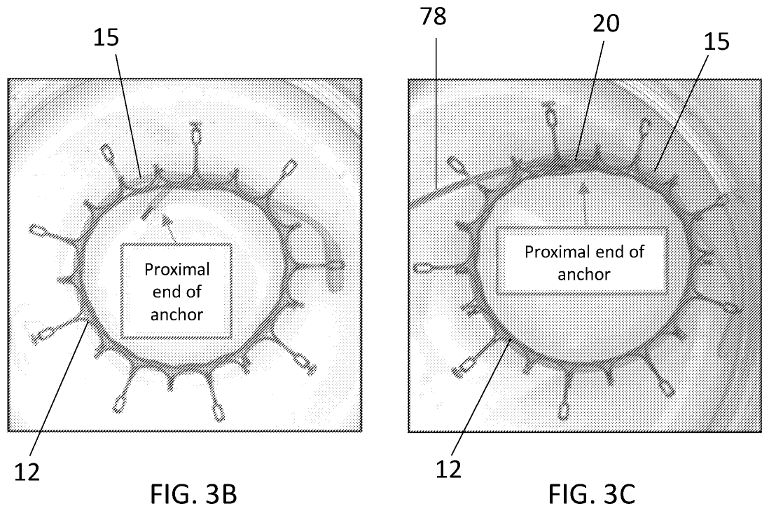

FIG. 3A shows a high-level diagram of an exemplary tethering system for the anchor The tether 78 is configured to be releasably secured to the anchor 15 by a connector 20. In some cases, the tether 78 may include a cable (also referred to herein as a wire) housed within a tubular housing 301. The inner cable can be configured to engage with the anchor 15 to keep the anchor 15 coupled to the tether 78. The tubular housing 301 may be stiff enough for the user to have control over the positioning of the anchor 15, yet have enough lateral flexibility to bend as it traverses the patient's vessels and/or heart. The tether 78 can have enough tensile strength to allow the user to deploy the anchor 15 (e.g., from an anchor delivery catheter) and/or to (e.g., subsequently) retract the tether 78 (e.g., into a valve delivery catheter). In some cases, the tether may be configured to allow the user to rotate (spin) the anchor 15. The tether 78 (including the tubular housing 301) may have enough tensile strength to resist axial compression and/or elongation as pulling and/or pushing forces are applied to the tubular housing 301 when manipulating the anchor 15. In some examples, the tether 78 is configured to elongate less than about 1 mm with an applied tensile load of five pounds. In some examples, the tether 78 may be able to withstand at least 15 pounds of tensile load at failure. In some examples, the tether 78 may be able to withstand between 15 and 20 pounds of load at failure.

The tether 78 may have a small enough diameter for entry into the patient's vessels and/or heart yet be resistant to kinking as it traverses through the patient's vessels and/or heart. In some examples, the tether 78 is configured to resist kinking with a minimum bend radius of 3 mm. A maximum outer diameter of the tether 78 may be designed considering the diameters of one or more surrounding catheters (e.g., as part of the anchor delivery system and/or the valve delivery system). For example, in some implementations, the tether 78 is the innermost member in a multiple catheter assembly (e.g., four-catheter assembly). In some examples, a maximum diameter of the anchor delivery catheter and/or the valve delivery catheter is 28 Fr, and a maximum outer diameter of the tether 78 is 0.07 inches. In some examples, the outer diameter of the tether 78 is at least a large as the outer diameter of the anchor 15. In some examples, a diameter of at least a distal portion of the tether 78, including the connector 20, may range between any two of the following values: 0.03 inches, 0.04 inches, 0.05 inches, 0.06 inches, and 0.07 inches.

The tether 78 (e.g., including the housing 301 and/or the cable housed therein) may be made of any of a number of materials. In some examples, the housing 301 and/or cable may comprise one or more of the following materials: stainless steel (e.g., 304 and/or 316 stainless steel), nickel titanium alloy (e.g., nitinol), titanium, cobalt chromium nickel alloy (e.g., Elgiloy®), nickel cobalt alloy (e.g., MP35N), cobalt chromium alloy, and one or more polymers such as a block copolymer comprising polyamide and polyether (e.g., Pebax®).

A distal end of the tether 78 may be coupled to a proximal end of the anchor 15 via the connector 20. The connector 20 may be a continuation of the housing 301, or one or more components coupled to the housing 301. The connector 20 may be configured to secure the anchor 15 to the tether 78 and release the anchor 15 at a prescribed time so that the anchor 15 may be freed from the tether 78 when the tether 78 is no longer needed. For example, the connector 20 can be configured to keep the anchor 15 connected to the tether 78 across procedural loading conditions, such as during delivery of the anchor 15 to the native valve and/or delivery of the prosthetic valve over the tether 78. The connector 20 may be configured to switch between a “locked” state, where the connector 20 secures the anchor 15 to the tether 78, and an “unlocked” state, where the anchor 15 and the tether 78 are decoupled from each other so that the anchor 15 can be separated from the tether 78.

In some cases, the connector 20 may include an opening that is sized and shaped to accept a proximal end of the anchor 15. The anchor 15 may include one or more engagement features (also referred to herein as retention features) that is/are configured to engage with one or more corresponding engagement features (also referred to herein as retention features) of the connector 20. For example, the connector 20 may include one or more engagement features within the inner lumen of the connector 20.

In some cases, the connector 20 may be configured to maintain the anchor 15 in a radially- and axially-fixed position such that the anchor 15 does not rotate or move distally/proximally relative to the connector 20. This can provide adequate control for positioning of the anchor 15 with respect to the patient's native valve (e.g., around the chordae tendineae). This can also provide adequate control to aid in visualizing the anchor 15, for example, using fluoroscopy and/or echo imaging techniques.

A proximal end of the tether 78 can include an actuator 21 that is configured to actuate one or more aspects of the connector 20 at a prescribed time. For example, the actuator 21 may be configured to switch the connector 20 between the locked and unlocked states to actuate retention and/or release of the anchor 20. In some examples, the connector 20 remains in a locked state until the actuator 21 is actuated to unlock the connector 20.

FIGS. 3B and 3C show an example anchor 15 wrapped around a frame structure 12 for a prosthetic valve, illustrating a frictional affect that the anchor 15 and tether 78 may experience. FIG. 3B shows the anchor 15 taking on a circular shape as it wraps around the frame structure 12 without being attached to a tether. FIG. 3C shows the anchor 15 still wrapped around the frame structure 12, but having a preload induced by the tether attached thereto by a connector 20. In some examples, a natural shape (e.g., pre-formed memory shape) of the anchor may have a somewhat smaller diameter than the diameter of the frame structure 12, thereby exerting a radially inward tension against the frame structure 12. This is demonstrated in FIG. 3B by the tendency of a proximal end of the anchor 15 to move radially inward. Note that normally the frame structure 12 can include a covering (e.g., fabric) that would prevent the proximal end of the anchor 15 from passing radially through the frame structure 12 as shown in FIG. 3B. As the frame structure 12 is expanded into the anchor, there may be an excess friction to remove the tether 78 from the anchor 15. The connector 20 may be configured to overcome friction effects from interaction of the anchor 15 with the tether 78 and/or prosthetic valve (e.g., frame structure 12) caused by the inward radial force exerted by the anchor 15. Thus, the connector 20 may be configured to provide a robust enough connection between the anchor 15 and the tether 78 to allow positional adjustment of the deployed anchor 15 via movement (e.g., pulling and/or pushing) of the tether 78, yet provide clean a separation of the tether 78 from the anchor 15 despite the frictional effects caused by the inward radial force anchor 15 (e.g., at the proximal end of the anchor 15).

In some examples, it may be desirable for some or all of the tether 78 to have a diameter that is within an upper limit. This can assure that the tether 78 can fit within an anchor delivery catheter (e.g., sheath 50 in FIGS. 1C-1F) and provide an atraumatic valve placement procedure. This may also allow for easier maneuverability of the tether 78 and/or anchor 15. In some examples, a diameter of at least a distal portion of the tether 78, including the connector may range between any two of the following values: 0.03 inches, 0.04 inches, 0.05 inches, inches, and 0.07 inches.

The tether 78 and connector 20 may configured to retain the anchor 15 across complex procedural loading conditions (e.g., during delivery and positioning of the anchor 15 in the patient's heart and/or during delivery of the frame structure 12 over the tether 78). The connector 20 may be configured to be provide robust engagement during torsion and bending when manipulating the position of the anchor 15. For example, in some cases it may be desirable to position the anchor 15 in a planar position with respect to the native valve and/or at a particular axial height during deployment of the prosthetic valve. In some case, the tether 78 and connector 20 may configured to guide the anchor 15 without substantially affecting a planarity, footprint (e.g., diameter), and/or concentricity of the anchor 15. The actuator (e.g., actuator 21 in FIG. 3A) may be configured for simple actuation on the proximal end.

In any of the connection assemblies and mechanism described herein, the connector may be part of (or coupled to) the tether 78 or the anchor 15. In some cases, the connector 20 is part of (or coupled to) the tubular housing 301. In some cases, the connector 20 includes a first portion that is part of (or coupled to) the tether 78, and a second portion that is part of (or coupled to) the anchor 15. In some cases, the connector 20 is a separate piece that is coupled to the tether 78 and/or the anchor 15 to attach the anchor 15 to the tether 78.

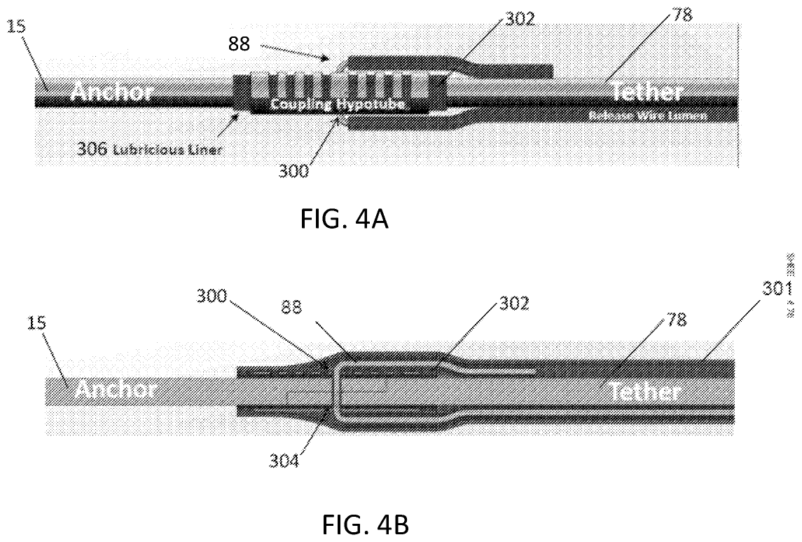

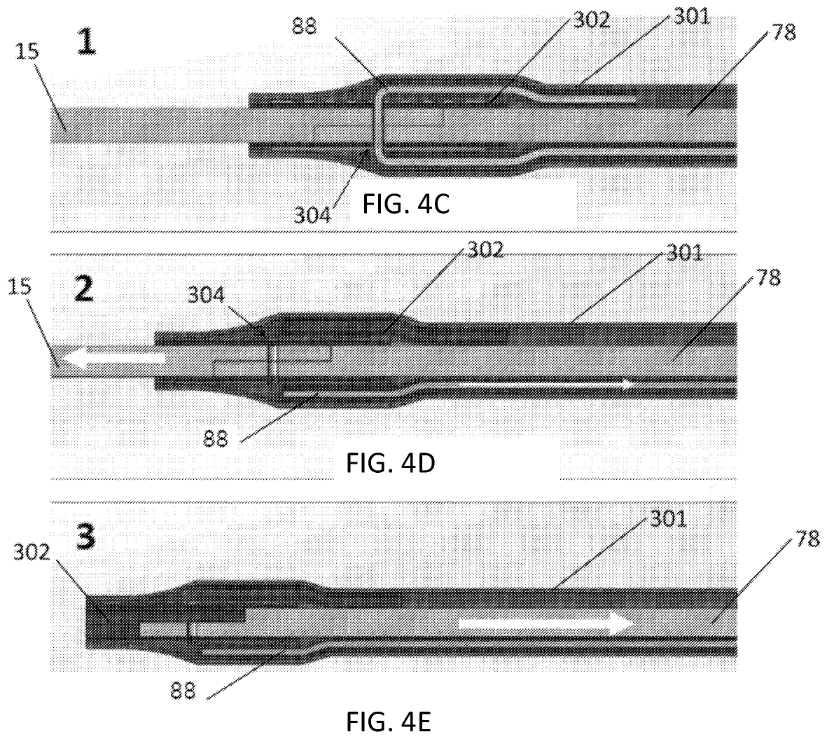

FIGS. 4A and 4B show various views of an exemplary anchor 15 coupled to a tether 78 with a connector 300 between the anchor 15 and the tether 78. FIG. 4A is a side view. FIG. 4B shows a side cross-sectional view. The tether 78 may comprise a (e.g., round) wire (also referred to as a cable) housed in a tubular housing 301. The housing 301 may, for example, comprise a mesh. The mesh may comprise a wire or polymer mesh. A distal end of the tether 78 may comprise a tube 302 bonded to the housing 301 (e.g., mesh). The tube end 302 of the housing 301 may provide support to the connection point 300 between the anchor 15 and the tether 78. The coupling tube 302 may comprise a lubricous liner 306 therein to facilitate decoupling of the anchor 15 and the pusher arm 78. The wire (or wires) of the tether 78 may be substantially similar in cross-section and structure to the anchor 15 in order to facilitate overlap of the layers and provide support. The tether 78 may be coupled to the anchor 15 by a retention wire 88. The retention wire 88 may run the length of the tether 78 from the connection point 300 to the proximal end. A distal end of the retention wire 88 may be threaded through a hole 304 spanning at least a portion of both the anchor 15 and the tether wire 78 when aligned in the delivery configuration. When threaded as such, the retention wire 88 may act as an engagement pin to keep the anchor 15 and tether wire 78 together during deployment of the anchor 15. Once the anchor 15 is deployed, the proximal end of the retention wire 88 may be translated proximally in order to disengage the retention wire 88 from the anchor 15 and release the anchor from the tether 78. The proximal end of the retention wire 88 may be coupled to an actuation mechanism in/on the delivery device in order to translate the retention wire 88. Alternatively, the proximal end of the retention wire 88 may be manually manipulated by, at, or near a proximal end of the delivery device in order to translate the retention wire 88. Once disengaged, the tether 78 may be retracted from the body, leaving the deployed anchor 15 adjacent the native valve as described herein.

FIGS. 4C-4E show sequential views of the release of the anchor 15 from the tether 78. FIG. 4C shows the anchor 15 coupled to the tether 78 (e.g., during deployment of the anchor 15 to the heart). After the anchor 15 has encircled the one or more structures of the native valve, the inner shaft 52 has been retracted, the frame structure 12 has been deployed, valve positioning has been confirmed, and/or hemodynamics have been assessed, the anchor 15 may be decoupled from the proximal pusher 78. As shown in FIG. 4D, actuation of the release mechanism (e.g., proximal translation of the retention wire 88) may disengage the retention wire 88 from the hole 304. As shown in FIG. 4E, the tether 78 may then be retracted into the delivery device 301 (e.g., housing) and de-coupled from the anchor 15 (via decoupling at the connector 300).

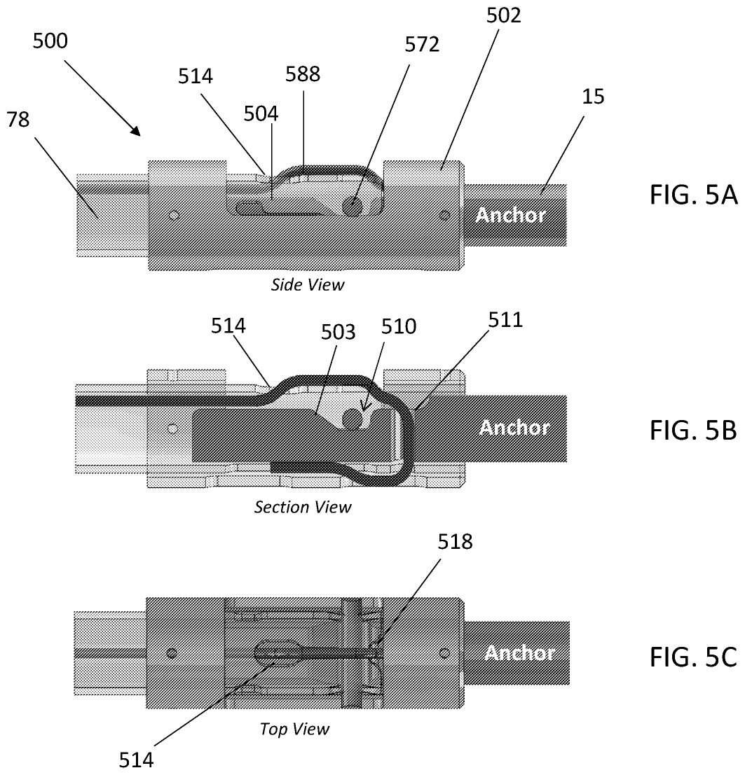

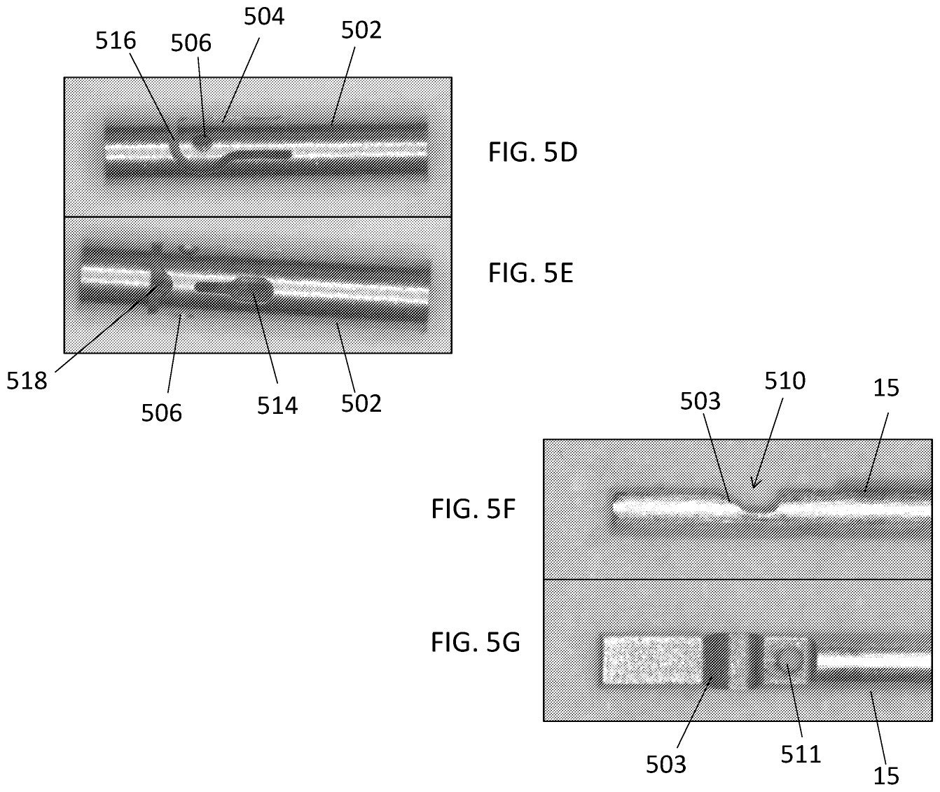

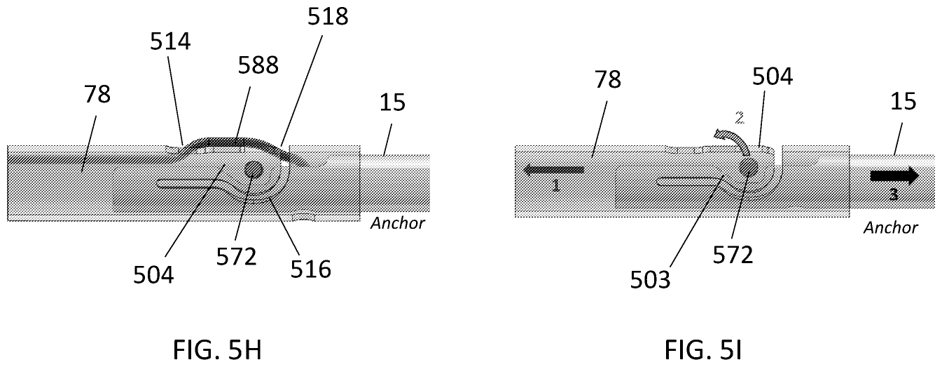

Another exemplary connector 400 for a tether 78 and anchor 15 is shown in FIGS. 5A-5I. Similar to connection mechanism of FIGS. 4A-4E, a distal end of the tether 78 may include a tube 502 bonded (or welded, crimped, soldered, or swaged) to the tether 78. Unlike the connector 300 of FIGS. 4A-4E, the tube 502 can include a release latch 504 therein configured to engage with a ramp 503 on the proximal end of the anchor 15. A retention cross-pin 572 can be attached to the tube 502 across a recess 510 within the anchor 15 to entrap the cross-pin 572 and to rotationally lock the anchor 15 and the tether 78. Rotationally locking can substantially prevent the anchor 15 from rotating with respect to the tether 78 (e.g., from rotating about the axis of the tube 502). Preventing rotation of the anchor 15 relative to the tether 78 can advantageously ensure that the anchor 15 remains in a stable orientation during deployment of a valve therein.

Additionally, the tether 78 may be coupled to the anchor 15 by a retention wire 588 that runs the length of the tether 78. A distal end of the retention wire 588 may be threaded through a first hole 514 of the tube 502, a second hole 518 of the tube 502, and a hole 511 of a proximal engagement portion of the anchor 15, thereby spanning at least a portion of both the anchor 15 and the tether 78. This configuration can frictionally engage the retention wire 588 with the tube 502 and anchor 15. In this way, the retention wire 588 can prevent the release latch 504 from opening by impeding the latch 504. Further, the release latch 504 can advantageously enable a reduced profile of the tube 502 relative to the retention wire 588, thereby minimizing the diameter of the connector 500.

FIGS. 5D and 5E show detailed views of the tube 502, which is coupled to the tether 78 as shown in FIGS. 5A-5C. As shown, the release latch 504 may be a deflectable tab that is defined by a slot 516 cut (e.g., laser cut) into the tube 502. The retention wire 588 can prevent opening of the release latch 504 9By double shear when the release wire 588 is threaded through the first hole 514 and the second hole 518 of the tube 502. In this case, the first hole 514 can have an elongate shape having a wide section and a narrow section, where the narrow section may better engage with the retention wire. The second hole 518 can be contiguous with (e.g., be part of) the slot 516. The release latch 504 can include openings 506 on opposing sides of the tube 502 for inserting a retention cross-pin 572. The retention cross-pin 572 may be secured to the tube 502, for example, using a weld (e.g., laser weld).

FIGS. 5F and 5F show detailed views of the engagement portion at a proximal end of an anchor 15. The proximal end of the anchor 15 can be configured to be inserted within the lumen of the tube 502 in FIGS. 5D and 5E. The engagement features can be configured to releasably engage with the release latch 504 of the tube 502. A recess 510 cut into the side of the proximal end of the anchor 15 forms a ramp 503 having a sloped profile that can engage with and deflect the deflectable release latch 504. The hole 511 can be used to capture redundant release wire length. Note that the engagement features of the proximal end of the anchor 15 may be agnostic to other aspects of the anchor's geometry.

FIGS. 5G and 5I illustrate an exemplary release sequence of the connector 500. FIG. shows the anchor 15 engaged with the tether 78, where the retention wire 588 is threaded through the holes 514 and 518 of the tube 502 such that a portion of the retention wire 588 is radially outside the release latch 504. In this state, the retention wire 588 can frictionally engage with the tube 502 to prevent release of the anchor 15 from the tether 78.

To release the tether 78 from the anchor 15, the retention wire 588 can be pulled proximally into the tether 78, as shown in FIG. 5I. The tether 78 can then be pulled in a proximal direction (indicated by arrow 1), causing the release latch 504 to deflect as the cross-pin 572 travels over and engages with the ramp 503, thereby disengaging the tether 78 fully from the anchor 15. The anchor 15 can then be released distally (indicated by arrow 3) with respect to the tether 78. In some embodiments, by changing the angle of the ramp 503 or the stiffness of the release latch 504, the release forces of the connector 500 can be finely tuned.

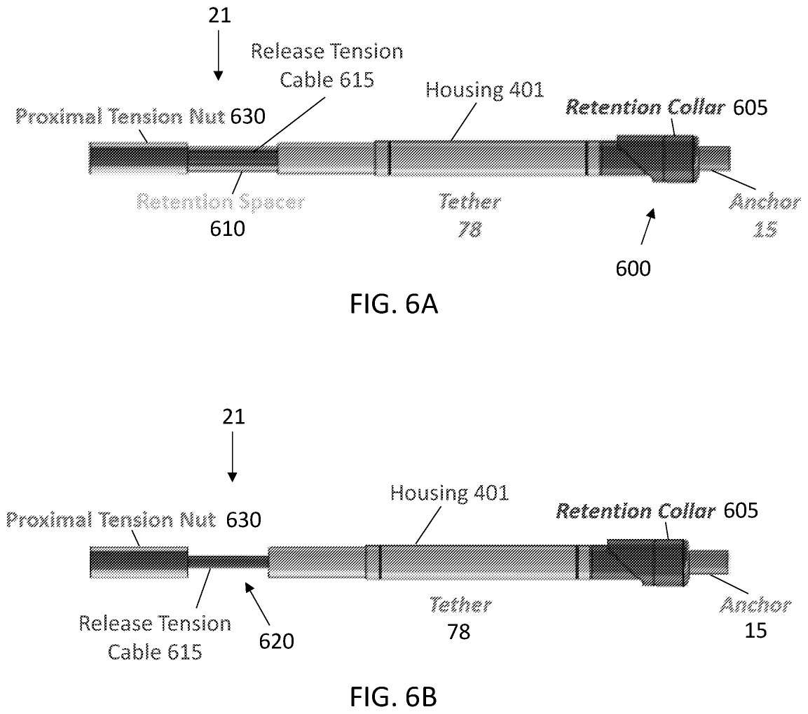

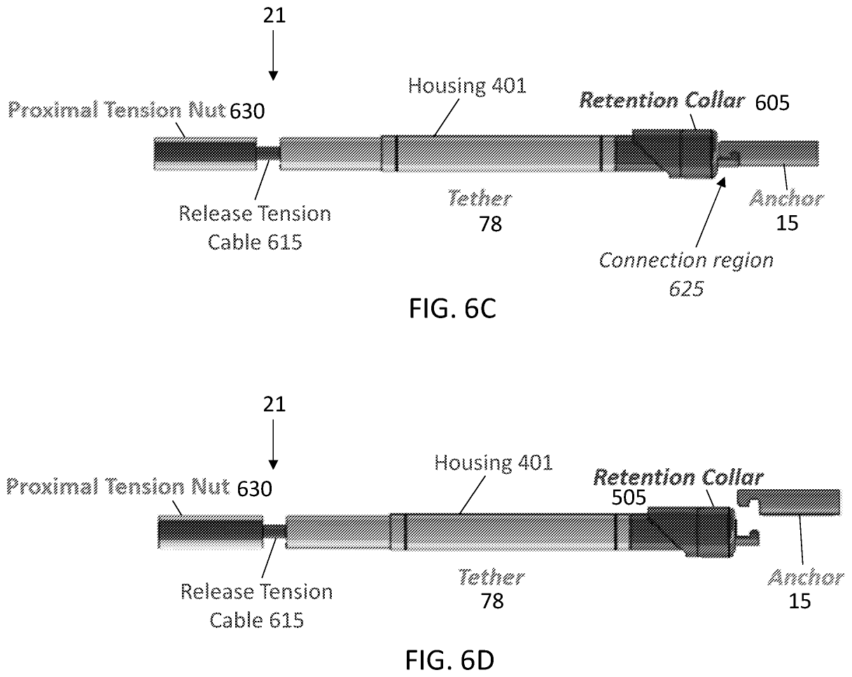

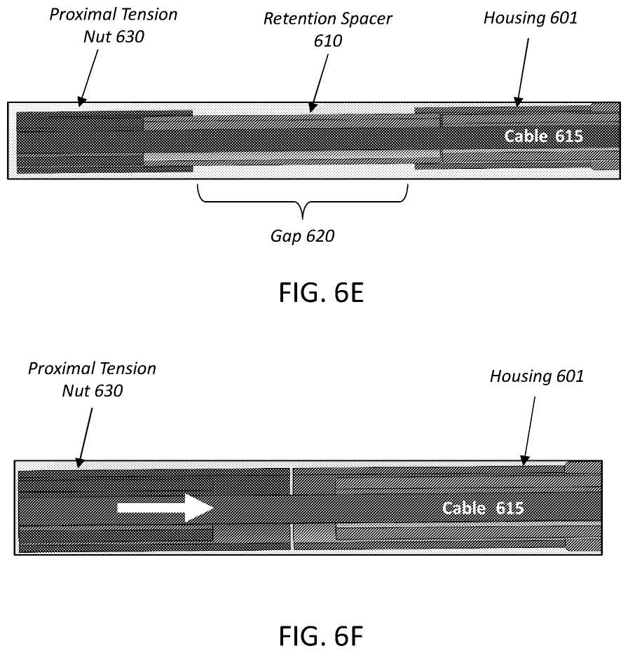

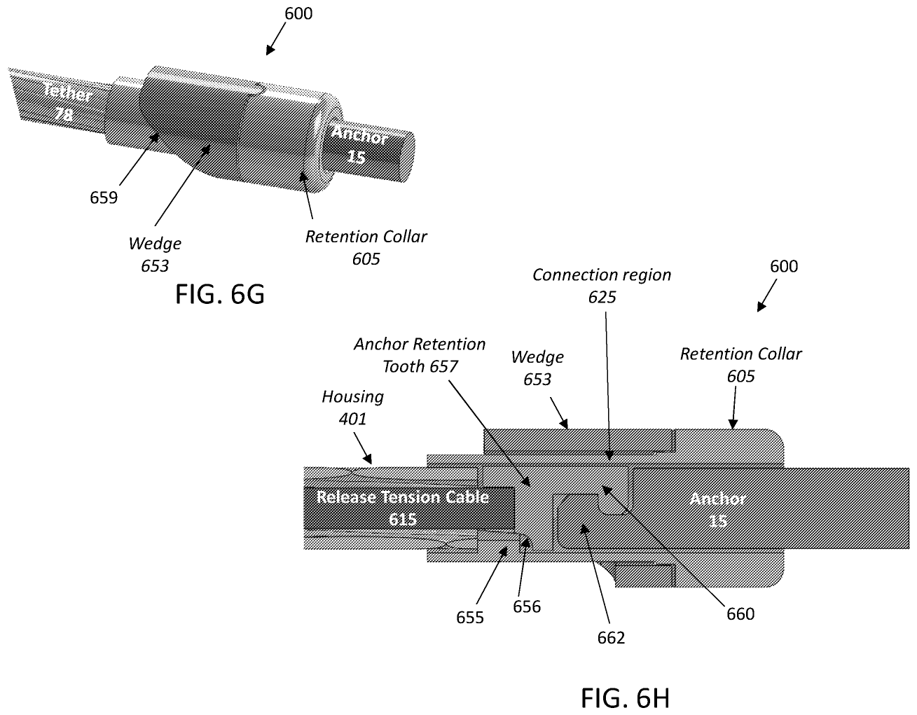

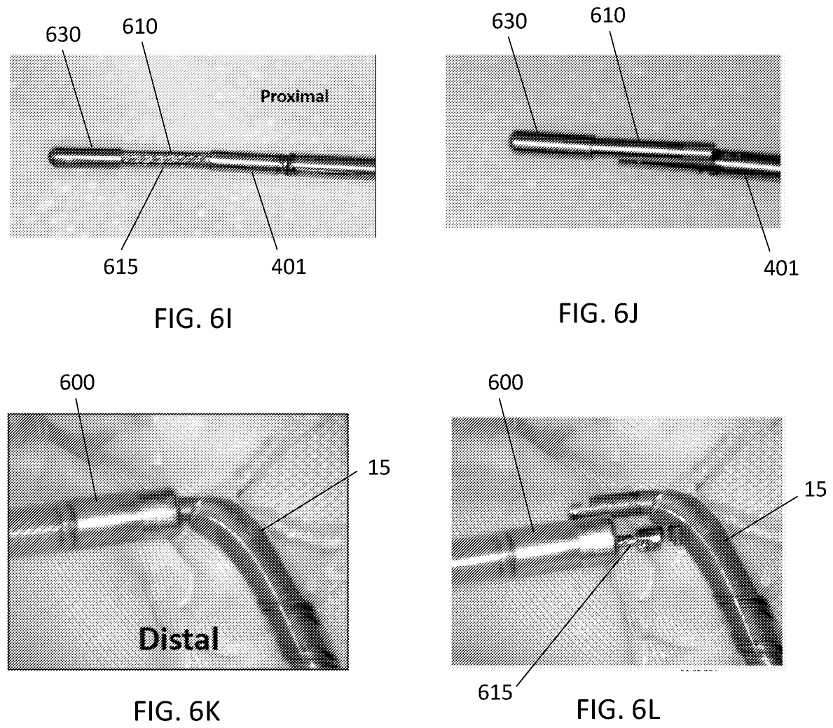

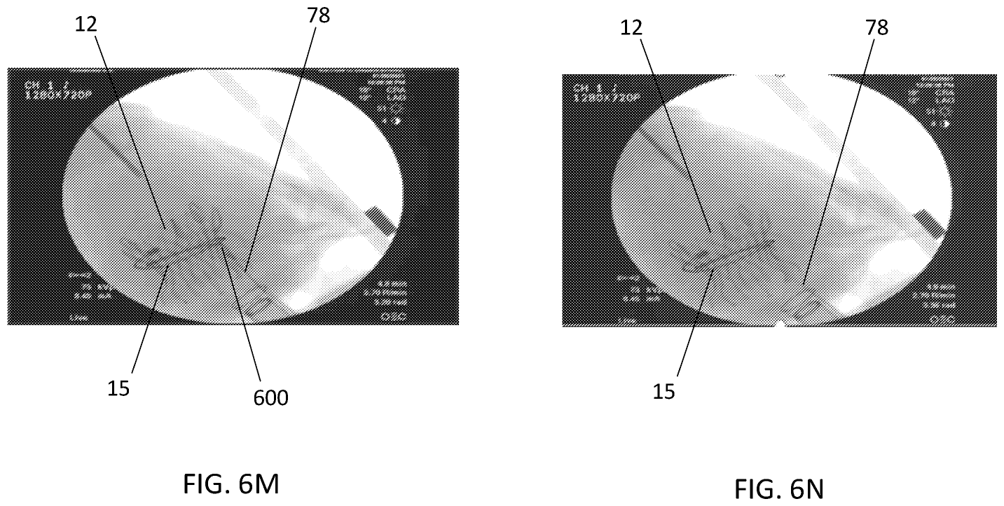

FIGS. 6A-6D show another exemplary connector 600, having a different connection mechanism. FIG. 6A shows an inner cable 615 coupled to the anchor 15 within the tubular housing 301. An actuator portion 21 at the proximal end of the tether 78 includes a removable retention spacer 610. The cable 615 can be held under tension when the retention spacer 610 is positioned within a gap between the tubular housing 301 and a proximal tension nut 630. This tension can keep the anchor 15 coupled to the tether 15 within a retention collar 605 portion of the tubular housing 301 at a distal end of the tether 78.

FIG. 6B shows the connector 600 after the retention spacer 610 is removed from the gap 620 between the tubular housing 301 and a proximal tension nut 630. Once the retention spacer 610 is removed, the gap 620 can progressively narrow as the proximal tension nut 630 and the cable 615 move distally. On the distal end of the tether, a connection region 625 between the tether 78 and the anchor 15 can exit distally from the retention collar 605, as shown in FIG. 6C. The connection region 625 can be configured to allow the anchor 15 to decouple from the tether 78 once the connection region 625 is no longer within the retention collar, as shown in FIG. 6D. Note that the cable 615 and the anchor 15 may be pushed distally or the collar 605 may be retracted proximally.

One advantage of the connection mechanism of connector 600 is that the distal end of tether 78, including the collar 605, can have a small profile (e.g., small diameter), which can be conducive to placement within the patient's vessels and/or heart.

FIGS. 6E and 6F illustrate section views of the proximal portion of the tether 78. FIG. 6E shows the retention spacer 610 positioned within the gap 620 between the housing 301 of the tether 78 and the proximal tension nut 630. The retention spacer 610 can put tension on the cable 615 and prevent the cable 615 from advancing. This corresponds to the connector 600 being in a locked state, where the anchor 15 is secured to the tether 78.

FIG. 6F shows the retention spacer 610 removed from the gap (620, FIG. 6E). This can allow the proximal tension nut 630 and the cable 615 to be advanced distally (e.g., by pushing) such that the connection region 625 between the tether 78 and the anchor 15 can move distally relative to the housing 301, thereby decoupling the anchor 15 from the tether 78. This corresponds to the connector 600 being in an unlocked state, where the anchor 15 can be released from the tether 78.

FIGS. 6G and 6H illustrate a retention and release mechanism of the connector 600 at the distal end of the tether 78. A distal end of the connector 600 can include a retention collar 605 having a tubular shape defining an inner lumen for housing the cable 615 and anchor 15 therein. The inner lumen of the collar 605 can be shaped and sized to radially surround the cable 615 and the anchor 15 at the connection region 625 to keep the cable 615 and the anchor 15 coupled to each other.

The cable 615 can be coupled to an anchor retention tooth 657 having a retention feature 660 that is shaped to interlock and engage with a corresponding retention feature 662 of the anchor 15. When the retention features 660 and 662 are mated within the collar 605, the anchor 15 can be prevented from rotating with respect to the cable 615. The inner walls of the collar 605 can include a shelf 655 that is configured to engage with a ledge 659 of the cable 615 (e.g., tooth 657) to prevent rotation of the cable 615 with respect to the collar 605. In this way, the collar 605 can be configured to fix rotational positions of the cable 615 and anchor 15 with respect to each other and the collar 605.

A wedge portion 653 of the connector 600 can include an angled edge 659 for compatibility with a valve delivery device or a component of the valve delivery device (e.g., a positioning tool). In this example, the wedge portion 653 can be configured to radially surround part of the outer surface of the collar 605.

The collar 605, the wedge 653 and tooth 657 may be made of material having sufficient tensile strength to maintain coupling of the tether 78 and anchor 15 during manipulation of the tether 78 and anchor 15. In some cases, the collar 605, the wedge 653 and/or tooth 657 may be made of one or more of the following materials: stainless steel (e.g., 304 and/or 316 stainless steel), nickel titanium alloy (e.g., nitinol), titanium, cobalt chromium nickel alloy (e.g., Elgiloy®), nickel cobalt alloy (e.g., MP35N), and cobalt chromium alloy.

FIGS. 61 and 6J show the proximal portion of the tether 78. FIG. 6I shows the retention spacer 610 (back side of the cable 615) positioned within the gap between the proximal tension nut 630 and the housing 301. FIG. 6J shows the retention spacer 610 removed from the gap. As shown, the retention spacer 610 may be coupled to the proximal tension nut 630 so that the spacer 610 may remain attached to the tether after removal from the gap.

FIG. 6K shows the distal portion of the tether 78 when the retention spacer 610 is positioned within the gap in the proximal portion of the tether 78 as shown in FIG. 6I. The proximal end of the anchor 15 is positioned within the connector 600 and engaged (locked) with the cable 615 therein.

FIG. 6L shows the distal portion of the tether 78 when the retention spacer 610 is removed from the gap in the proximal portion of the tether 78 as shown in FIG. 6J. The cable 615 has been advanced distally relative to the connector 600 so that the anchor 15 is released from the connector 600 and disengaged (unlocked) from the cable 615.

FIGS. 6M and 6N illustrate the connection mechanism of the connector 600 in action with a heart. The anchor 15, while secured to the tether 78 via the connector 600, is positioned around the native valve and secured to the native valve by engagement with the chordae tendineae. The tether 78 is then used to guide the frame structure 12 of the valve prosthesis toward the native valve, where the frame structure 12 is positioned within the native valve and expanded such that the spiral shaped anchor 15 surrounds the frame structure 12, as shown in FIG. 6M. After securement of the frame structure 12 within the native valve, the connector 600 can be unlocked to release the anchor 15 from the tether 78, and the tether 78 retraced as shown in FIG. 6N.

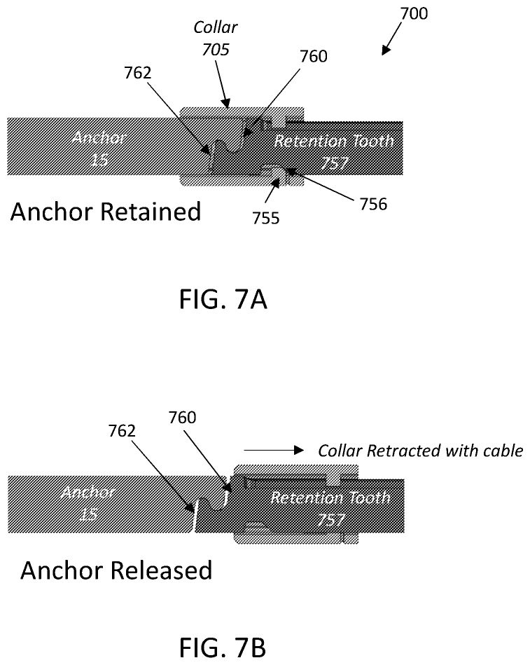

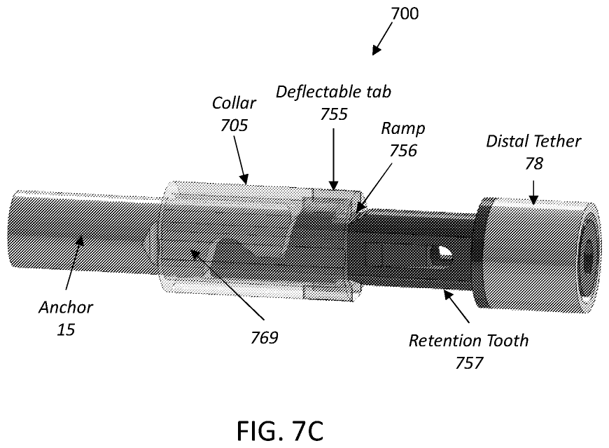

FIGS. 7A-7C illustrate another example connector 700, which is a variation of the connector 600. In this variation, the inner surface of the collar 705 can include a tab 755 that engages with a ramped recess of the tooth 757 to retain the anchor 15 within the collar 705 in the locked state, as shown in FIG. 7A. The ramped recess may be shaped to require a specific amount of force for the tab 755 to deflect over the ramp. In this way, the connector 700 can depend on a force-based release of the anchor 15, which may be more controllable than a distance-based release.

The retention tooth 757 and the anchor 15 have matching interface surfaces interface surfaces 760 and 762 that can keep the tooth 757 and the anchor 15 from rotating with respect to each other when engaged. In this case, the interface surfaces 760 and 762 can be at non-orthogonal angles relative to the central axis of the retention tooth 757 and the anchor 15. These angled interface surfaces 760 and 762 may allow for easy release of the anchor 15 from the retention tooth 757 when the retention features are advanced distally relative to the collar 705, as shown in FIG. 7B. That is, the angle of the interface surfaces 760 and 762 may prevent the anchor 15 and tooth 757 from binding once released out of the collar 705. Note that the retention tooth 757 and the anchor 15 may be pushed distally or the collar 705 may be retracted proximally (indicated by arrow in FIG. 7B).

FIG. 7C shows a perspective view of the connector 700, with the collar 705 shown as semitransparent for viewing therein. This figure shows a different view of the deflectable tab 755 of the collar 705 and the ramp 756 of the tooth 757. The inner surfaces of the collar 705 may also include flat surfaces 769 that engage with corresponding flat surfaces of the sides of the tooth 757 and anchor 15 to keep the tooth 757 and anchor 15 from rotating relative to the collar 705.

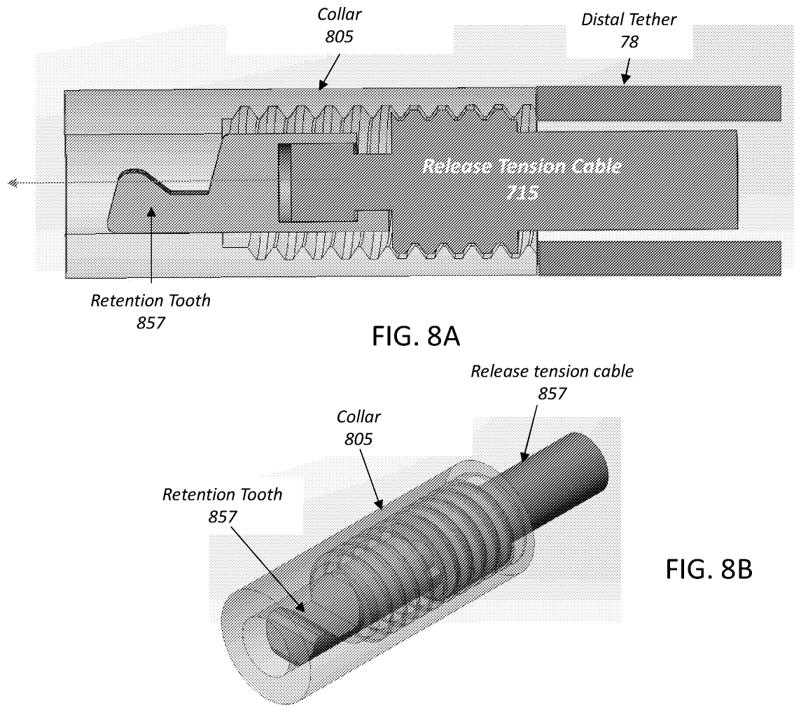

FIGS. 8A and 8B illustrate another example connector 800, in this case, having a screw-driven retracting collar. As shown, the internal walls of the collar 805 can have threads, and the exterior surface of the inner cable 815 can have corresponding threads. Rotating the cable 815 relative to the collar 805, or rotating the collar 805 relative to the cable 815, can cause the respective threads to engage and retain the cable 815 within the collar 805 in a locked position. Rotating the cable 815 relative to the collar 805, or rotating the collar 805 relative to the cable 815, further can cause the collar 805 to retract so that the tooth 857 can advance distally out of the collar 805 and release the anchor.

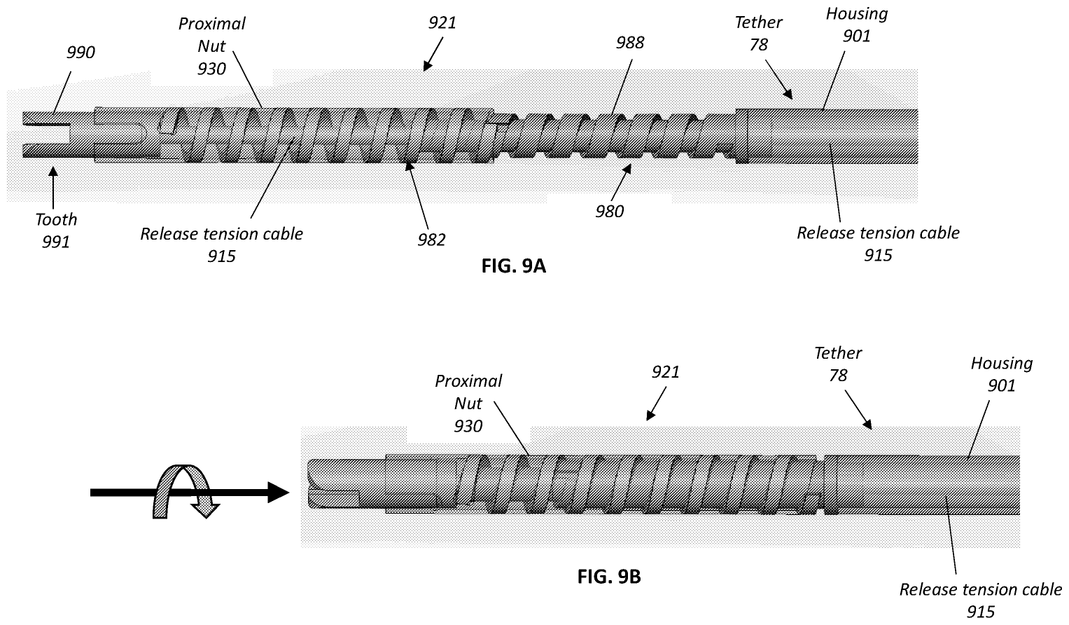

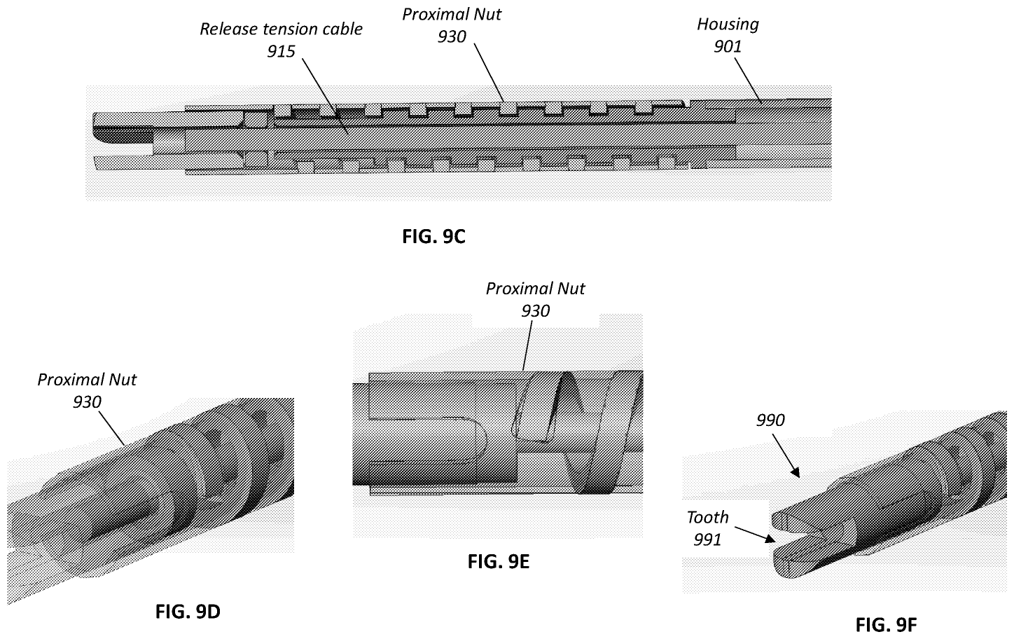

FIGS. 9A-9C illustrate another variation of an actuator assembly 921, in this case, having a lead screw configuration. The proximal end 988 of the housing 901 of the tether 78 has a threaded outer surface 980 that is configured to engage with a threaded opening 982 of a proximal nut 930. A proximal end 990 of the cable 915 can include a mating feature (e.g., tooth) that is configured to mate with a device or tool that facilitates rotation of the cable 915 relative to the housing 901. The cable 915 may be fixedly coupled to the nut 930, e.g., by weld.

FIG. 9A shows the actuator assembly 921 applying tension to the cable 915 such that connector at the distal end of the tether 78 is in a locked state and retains the anchor. Rotation of the cable 915 and nut 930 relative to the housing 901 (e.g., via the tooth 991) can cause the proximal nut 930 and the cable 915 to move distally, as shown in FIG. 9B, thereby causing the connector at the distal end of the tether 78 to release the anchor. In some cases, an outer surface of the tether 78 may have a maximum diameter. In some examples, the maximum diameter ranges between any two of the following values: 0.03 inches, 0.04 inches, 0.05 inches, 0.06 inches, and 0.07 inches. FIG. 9C shows a section view of the actuator assembly 921 in a locked state.

FIGS. 9D-9F show close-up views of the proximal end 988 of the housing 901, the proximal nut 930 and the mating feature 991 (e.g., tooth).

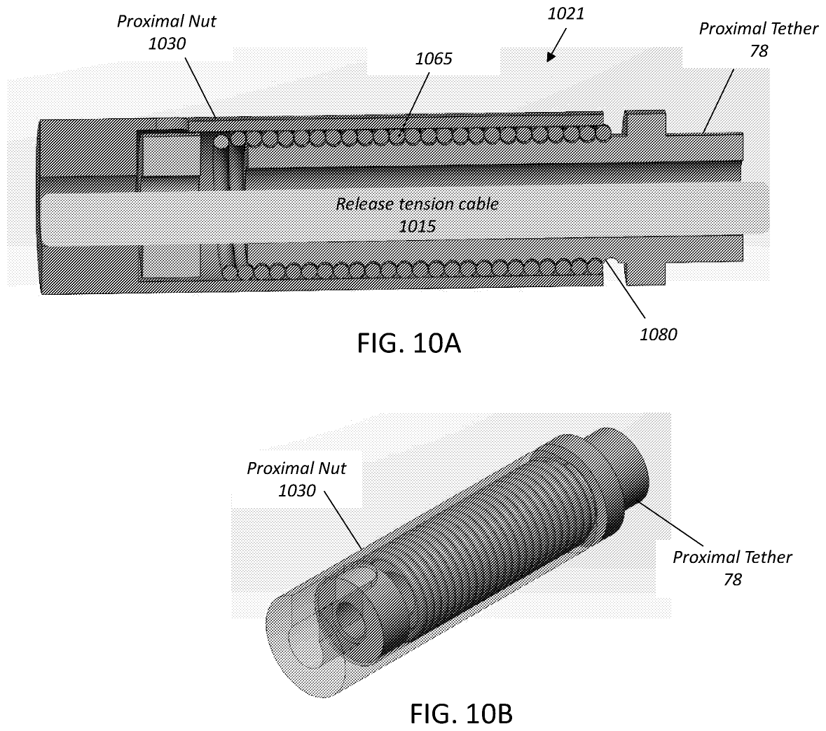

FIGS. 10A and 10B illustrate another variation of an actuator assembly 921 having a similar lead screw configuration as the actuator assembly 921 except that the internal threads within the proximal nut 1030 are formed by a coil 1065. In some cases, the external threads at the outer surface 1080 of the proximal end of the tether 78 can be formed from a ball end mill. This construction may have some manufacturability advantages over the actuator assembly 921.

In either of the screw-based actuator assemblies 921 and 1021, the pitch of the threads may vary depending on design requirements. For example, the pitch may be fine enough to allow the user to adequately control the release of the anchor but not too fine such that the user is encumbered by having to make too many turns of the proximal nut 930/1030.

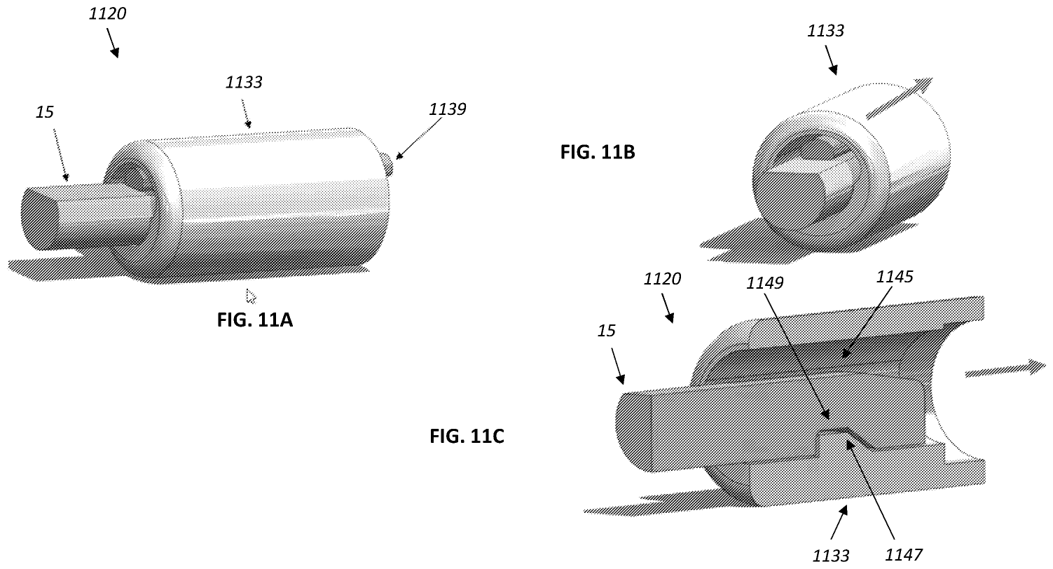

FIGS. 11A-11C illustrate another variation of connector 1120, in this case, having a removable shim configuration. A distal end of the housing 1001 of the tether can include a collar 1133 that has a tubular shape. The inner lumen of the collar 1133 can be configured to accept the anchor 15 and a retractable wire 1139. When in the locked state, the anchor 15 and the wire 1139 can be secured within the collar 1133 by frictional engagement. To release the anchor 15, the wire 1139 can be pulled proximally to remove the collar 1133 from the inner lumen of the collar 1133 and release the frictional engagement with the anchor 15. This gives the anchor 15 space 1145 within the inner lumen of the collar 1133 to move radially therein and be released from the collar 1133. Alternatively or additionally, the collar 1133 may be pulled proximally (e.g., by pulling the tether housing) past the distal end of the wire 1139 to disengage and release the anchor 15. This configuration can rely on a simple wire retraction actuator configuration and may not need a tension-based actuator (e.g., FIG. 6E, 6F, 8A-8B, 9A-9F, or 10A-10B).

In some cases, the inner walls of the collar 1133 includes a protruding feature 1147 that is shaped to mate with a recess 1149 in the anchor 15. These mating features 1147 and 1149 can provide further engagement of the anchor 15 to the collar 1133 to assure that the anchor 15 does not become released too easily. That is, this configuration can require that space 1145 be unoccupied by the retractable wire 1139 and that the anchor 15 be radially shifted to occupy the space 1145 before the anchor 15 can be release. The protruding feature 1147 may be ramped to facilitate the shift in movement of the anchor 15 into the space 1145. Note that in alternative examples, the collar 1133 can include a recessed feature that engages with a corresponding protruding feature of the anchor 15.

Any of the connectors described herein may be configured to withstand a tensile load high enough to prevent breakage of the connector (and undesired decoupling of the anchor and tether) during the various procedures of delivering the valve prosthesis. For example, the connector may be configured to withstand a tensile load ranging between any two of the following values: 7 lbs, 10 lbs, 15 lbs, 18 lbs, 20 lbs, 23 lbs, 25 lbs, and 30 lbs.

Additional embodiments of methods and devices for delivering an anchor/valve prosthesis are described in U.S. patent application Ser. No. 16/824,576, filed Mar. 19, 2020 and U.S. patent application Ser. No. 16/594,946, filed Oct. 7, 2019, the entire disclosures of which are incorporated by reference herein.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for anchoring a valve prosthesis, the system comprising:
an anchor comprising a wire having a shape and size to engage with chordae tendineae and/or leaflets of a native valve; and
a tether releasably coupled to a proximal end of the anchor, the tether configured to maintain or adjust a position of the anchor, the tether comprising a cable housed within a tubular housing, a distal end of the tubular housing having a connector with an opening sized and shaped to accept and engage with an engagement feature at the proximal end of the anchor, the connector configured to transition between a locked state to secure the anchor with the tether, and an unlocked state to decouple the anchor and the tether.

2. The system of claim 1, wherein the wire has a spiral shape configured to extend around a least a portion of the chordae tendineae.

3. The system of claim 1, wherein the wire is configured to extend around the valve prosthesis.

4. The system of claim 1, wherein a distal end of the cable comprises a corresponding engagement feature configured to engage with the engagement feature of the anchor.

5. The system of claim 4, wherein the connector is configured to radially surround the engagement feature of the anchor and the corresponding engagement feature of the cable when the connector is in the locked state.

6. The system of claim 1, further comprising an actuator at a proximal end of the tether, the actuator configured to transition the connector between the locked and unlocked states.

7. The system of claim 1, wherein the anchor is rotationally locked with respect to the tether when the connector is in the locked state.

8. The system of claim 7, wherein the anchor and the tether are rotationally locked with respect to the tubular housing when the connector is in the locked state.

9. The system of claim 1, wherein the anchor and the tether are rotationally locked with respect to the tubular housing when the connector is in a locked state.

10. The system of claim 1, wherein a diameter of the tether ranges between 0.03 inches and 0.05 inches.

11. The system of claim 1, wherein the connector comprises a retention wire that runs through the tubular housing and is threaded through an opening of the anchor to engage the tether with the anchor.

12. The system of claim 11, wherein the retention wire is configured to disengage from the anchor when pulled proximally.

13. The system of claim 1, wherein the tubular housing includes a release latch configured to frictionally engage with a retention wire, wherein the release latch is configured to deflect upon retraction of the retention wire to disengage the retention feature and release the anchor from the tether.

14. The system of claim 13, wherein the retention feature includes a recess configured to entrap a cross-pin running through the tubular housing.

15. The system of claim 14, wherein the cross-pin is configured to engage with a ramp defined by the recess.

16. The system of claim 1, wherein the tubular housing radially surrounds a distal end of the cable and the proximal end of the anchor when the connector is in the locked state.

17. The system of claim 16, wherein a retention feature of the anchor is configured to interlock with a corresponding retention feature of the cable within the tubular housing.

18. The system of claim 17, wherein the tubular housing is configured to radially surround the retention features of the anchor and the cable to maintain the anchor secured to the cable in the locked state.

19. The system of claim 17, wherein a proximal portion of the tether comprises a tension nut and a spacer, the spacer sized and shaped to fit between the tubular housing and the tension nut to form a gap therebetween and to apply a proximal tension on the cable, to retain the cable proximal to the connector at the distal end of the tubular housing when in the locked state.

20. The system of claim 19, wherein the spacer is configured to be removed from the gap to release the proximal tension such that the cable may be advanced distally past the connector and decouple the cable from the anchor.

21-44. (canceled)
